(19) United States
(12) Patent Application Publication
(10) Pub. No.: US 2007/0163604 A1
(43) Pub. Date: Jul. 19, 2007

(54) Leak test method for medical procedure via natural orifice

(75) Inventors: Takayasu Mikkaichi, Tokyo (JP); Kensei Nakahashi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., TOKYO (JP)

(21) Appl. No.: 11/331,968

(22) Filed: Jan. 13, 2006

(51) Int. Cl. A61B 19/00

(52) U.S. Cl. 128/898

(57) ABSTRACT

A leak test method for a medical procedure via a natural orifice includes: inflating the abdominal cavity by introducing a gas into the abdominal cavity via a conduit inserted from the abdominal cavity after closing an opening that communicates between the inside of a hollow organ and the inside of the abdominal cavity using an instrument inserted via the patient's natural orifice; monitoring no leakage between the hollow organ and the abdominal cavity of a fluid passing through the closed opening; and removing the conduit inserted into the abdominal cavity once no leakage of the fluid has been confirmed.

### BACKGROUND OF THE INVENTION

1. Field of the Invention

The present invention relates to confirming of the state of a closed opening after closing an opening that is formed in the wall of a hollow organ.

2. Background Art

Laparoscopic surgery is a conventionally known technique that has been employed when carrying out a medical procedure (including observation or treatment, with the term “medical procedure” including the same when used below) on the internal organs of the human body. Rather than making a large abdominal incision, laparoscopic surgery provides for the medical procedure to be carried out by making several openings in the abdominal wall, and inserting a rigid laparoscope and forceps or other such surgical instruments into these respective openings. Since only small openings are made in the abdominal wall, this type of surgery offers the benefit of being minimally invasive and, thus, hastening patient recovery.

As a method of even further reducing invasiveness to the patient, it has been proposed in recent years to carry out medical procedures by inserting an endoscope into the patient via a natural orifice such as the mouth, nostrils or anus. One example of such an medical procedure is disclosed in U.S. Pat. No. 5,458,131. In this method, a flexible endoscope is inserted via the mouth of the patient whose abdomen has been inflated with gas, and the endoscope is then introduced into the abdominal cavity via an opening made in the stomach wall. The endoscope is used as a monitor within the abdominal cavity. The organ is then treated using an instrument passed through the endoscope, and an instrument passed through another opening created in the stomach or passed in from the rectum through an opening formed in the sigmoid colon. Once the procedure inside the abdominal cavity is completed, the instrument is removed and the opening is closed. When closing the opening, the tissue around the opening is gathered together by suctioning, and then fastened with an O-ring.

### SUMMARY OF THE INVENTION

It is the objective of the present invention to provide a method for more easily confirming the state of a closed opening after closing of an opening present in the wall of a hollow organ that communicates between the inside of the hollow organ and the abdominal cavity.

A leak test method for a medical procedure via a natural orifice according to the present invention includes: inflating the abdominal cavity by introducing a gas into the abdominal cavity via a conduit inserted from the abdominal cavity after closing an opening that communicates between the inside of the hollow organ and the inside of the abdominal cavity using an instrument inserted via the patient's natural orifice; monitoring no leakage between the hollow organ and the abdominal cavity of a fluid passing through the closed opening; and removing the conduit inserted into the abdominal cavity once no leakage of the fluid has been confirmed.

### BRIEF DESCRIPTION OF THE DRAWINGS

FIG. 1A is a view in which a balloon has been passed into the endoscope.

FIG. 1B is an enlarged view of the end of the balloon and endoscope.

FIG. 2 is a view illustrating the medical procedure in the present embodiment, showing the patient in the reclined position.

FIG. 3 is a view in which the endoscope is inserted into the stomach.

FIG. 4 is a view illustrating the technique for treating a target site using a forceps passed through the working channel.

FIG. 5 is a view in which the endoscope has been withdrawn back into the stomach, and the opening is closed.

FIG. 6 is a view in which the entrance to the duodenum has been closed with a balloon.

FIG. 7 is a view in which water is supplied to the inside of the stomach so as to submerge the closed area.

FIG. 8 is a view showing the case where the inflating needle within the stomach is penetrated into the abdominal cavity.

FIG. 9 is a view showing the case where gas is infused into the abdomen by passing a testing tube through a part of the closed opening.

FIG. 10 is a view showing the case where a leak test is performed-using a pressure sensor provided on the inflating needle side.

### PREFERRED EMBODIMENTS

Embodiments according to the present invention will now be described below. Structural elements that are equivalent in the following discussion will be assigned the same numeric symbol and redundant description thereof will be omitted.

### First Embodiment

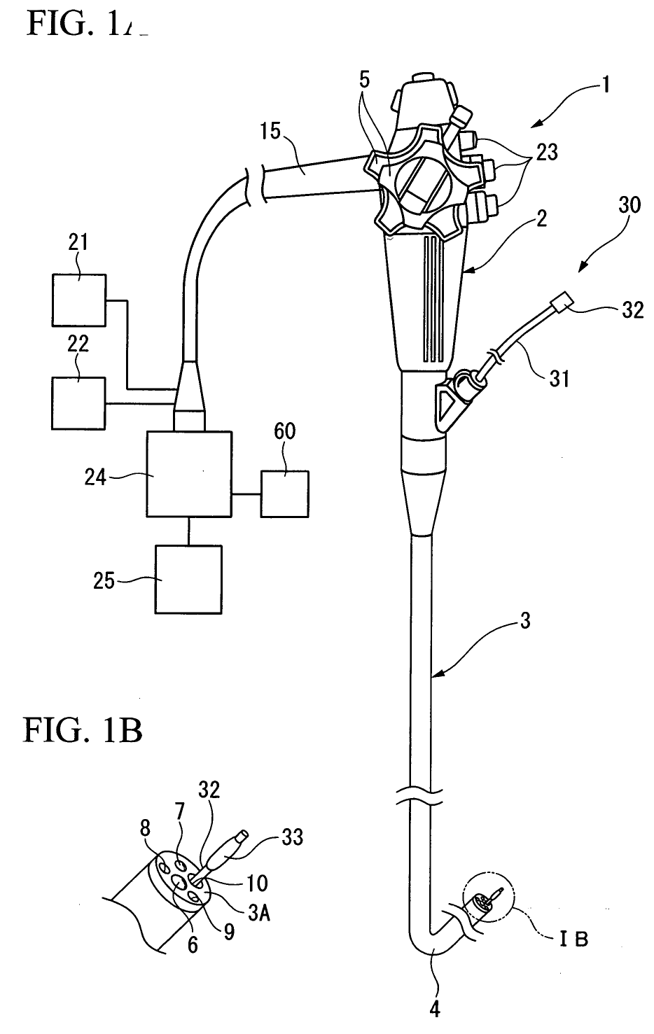

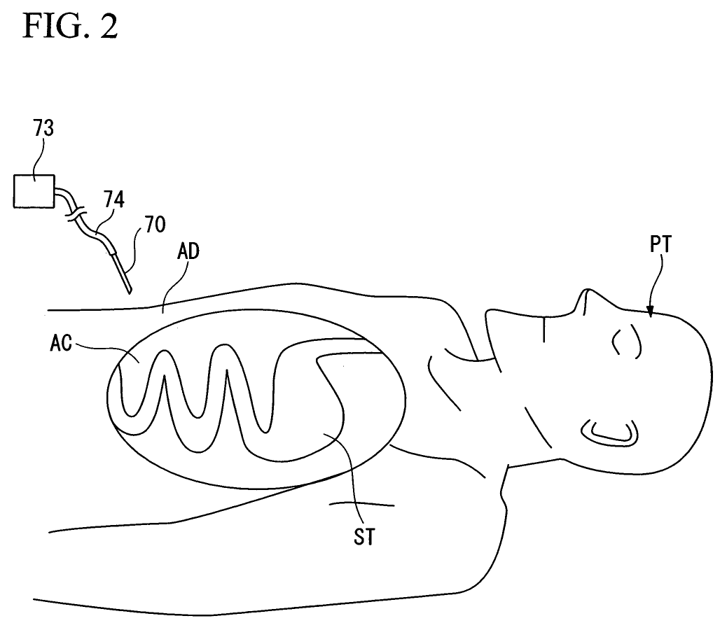

The flexible endoscope (referred to simply as “endoscope” hereinafter) employed in this embodiment is shown in FIGS. 1A and 1B. An endoscope 1 is provided with an inserted part 3 that extends out from an operating part 2, which is manipulated by the operator, and is inserted into the patient's body. The inserted part 3 is long and flexible. The end 4 of the inserted part 3 can be bent by operating an angle knob 5 that is disposed to the operating part 2. As shown in FIG. 2, an observation device 6 that observes inside the body and an illuminating device 7 are disposed to the end surface 3A of the inserted part 3. The observation device 6 is composed, for example, of an observation optical system such as an objective lens or the like, and a CCD (Charge Coupled Device), used as an image pickup element. The illuminating device 7 is designed to guide light from a light source device outside the body via an optical fiber. It is also acceptable to provide a design in which a signal output from the image pickup element is transmitted to a control device 24, described below, after passing through a signal line that passes within the inserted part 3. Further, it is also acceptable for the output signal from the image pickup element to be sent to the control device 24 wirelessly. In addition, it is also acceptable to provide the illuminating device 7 with a design that employs a conventionally known light emitting element (a light emitting diode, for example).

The end openings of the various types of channels 8-10 are provided to the end surface 3A of the inserted part 3. The gas-and-fluid introducing channel 8 is a conduit employed for supplying a fluid into the body. A suction channel 9 is a conduit for suctioning fluid from the body. A working channel 10 is a channel for passing instruments. These various types of channels 8-10 extend from the inserted part 3 toward the operating part 2, however their design is not limited thereto. For example, it is acceptable to omit the suction channel 9, and instead employ the working channel 10 for suctioning.

The opening on the base side of the working channel 10 is provided to the side of the operating part 2. The other channels 8 and 9 are connected to a gas-and-water introducing device 21 and a suction device 22 via a universal cable 15 shown in FIG 1A. The operations of introducing gas and/or fluid and suctioning can be controlled by buttons 23 provided to the operating part 2. Note that the endoscope 1 is connected to a control device 24 via the universal cable. The control device 24 is a device that controls the endoscope 1, and is equipped with an image processing device and a light source. The control device 24 can output various images (image) to a monitor 25.

In FIG. 1A, as one example of an instrument, a balloon 30 has been passed through the working channel 10 of the endoscope 1. The balloon 30 has a long and flexible sheath 31. The sheath 31 has a hollow formed internally. The end of this sheath 31 is pulled out from the operating part 2, and has a base 32 attached. A syringe (not shown) can be attached to this base 32, and fluid can be supplied to the hollow inside the sheath 31 via the syringe. The hollow is closed at the end of the sheath 31 and a bougie 33 is attached to the outer periphery. The bougie 33 communicates with the hollow inside sheath 31, and can be inflated by supplying the fluid.

The operation of this embodiment will now be described. As an example of a medical procedure performed prior to the leak test, the following descriptions will be directed to a medical procedure for treating an organ or tissue (referred to as the “target site” hereinafter) where the desired medical procedure is to be performed, by inserting an endoscope 1 via the patient's mouth, which is a natural orifice. A description of the leak test will then be made. However, the natural orifice into which the endoscope 1 is inserted is not limited to the mouth, but may be, for example, the nostrils or the anus. In addition, the treatment performed in the medical procedure includes various actions such as suturing, observation, excision, cellular collection, organ removal, etc.

As shown in FIG. 2, the patient PT is placed on his or her back, so that the abdomen AD is located on the top. The inflating needle 70 is then inserted into the abdomen AD, and a carbon dioxide gas or the like is introduced into the abdominal cavity AC via the respective conduits of inflating tube 74 and inflating needle 70 from inflating device 73, thereby inflating the abdominal cavity. When carrying out a medical procedure inside the abdominal cavity AC, it is preferable to inflate the abdominal cavity AC as a means of maintaining a space interval within the abdominal cavity. However, it is not absolutely essential to inflate the abdominal cavity if the desired space can be otherwise ensured. Further, the method for inflating the abdominal cavity is not limited to inflation with gas. Namely, it is also acceptable to employ conventionally known methods such as the suspension method to secure this space. In addition, it is also acceptable to inflate the abdominal cavity after the device (endoscope 1, for example) has been introduced into the abdominal cavity.

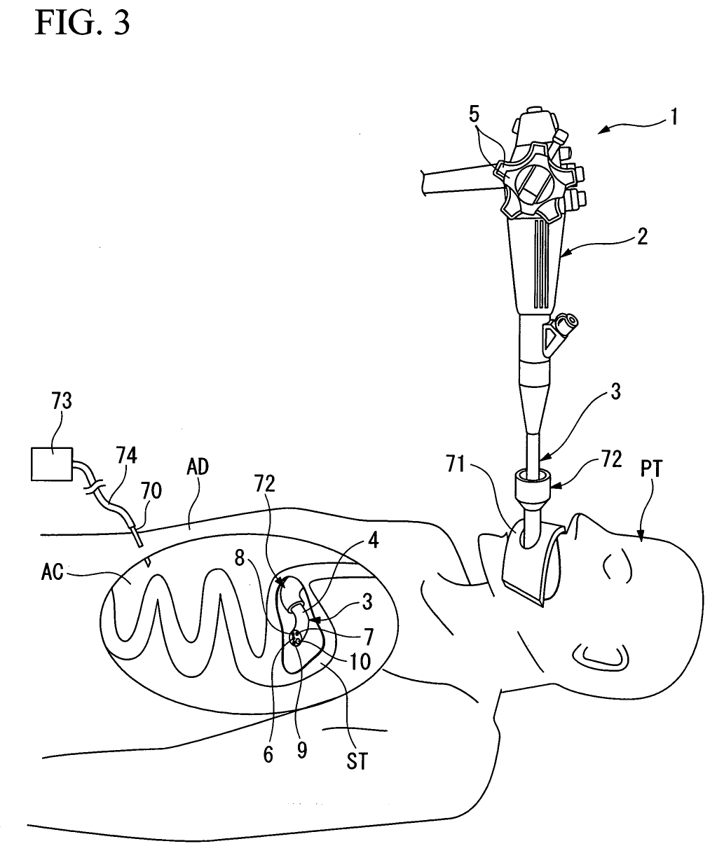

As shown in FIG. 3, once the abdomen AD of the patient PT has been inflated by infusing gas into the abdomen, the endoscope 1 is inserted into the body via a mouth piece 71 which is attached to the mouth of the patient PT. Preferably, as shown in FIG. 3, an over-tube 72 is concurrently employed when inserting the endoscope 1 into the body. The over-tube 72 is employed as a guide tube for inserting endoscope 1 or other such device that has an inserted part, into the body. However, it is also acceptable to directly insert the device into the body without employing an over-tube 72. Once the inserted part 3 reaches the stomach ST, gas is sent from the gas-and-water introducing channel 8, to inflate stomach ST. An instrument for cutting, such as a high frequency knife for example, is passed through the working channel 10, and an opening is formed by incising the stomach wall. The incised area is preferably the anterior wall of the stomach.

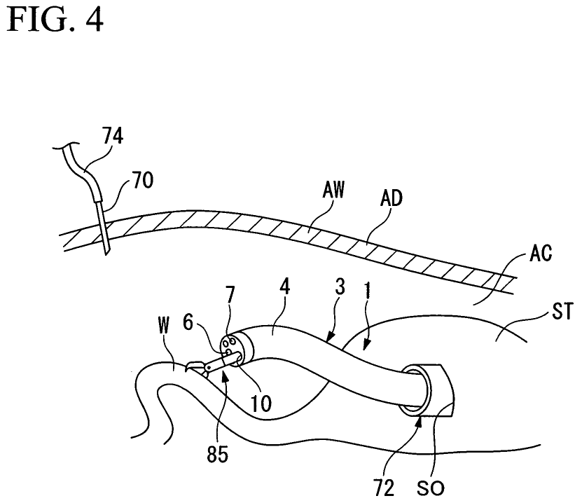

Once an opening SO communicating with the abdominal cavity AC is formed in the stomach wall, the inserted part 3 is introduced into the abdominal cavity AC from the opening SO As shown in FIG. 4, the forceps 85 are passed into the working channel 10 in place of the high-frequency knife, to treat the target site W. Since the technique within the abdominal cavity AC is completed at this point, the endoscope 1 and the over-tube 72 are withdrawn, and the inserted part 3 is withdrawn from the abdominal cavity AC into the stomach ST.

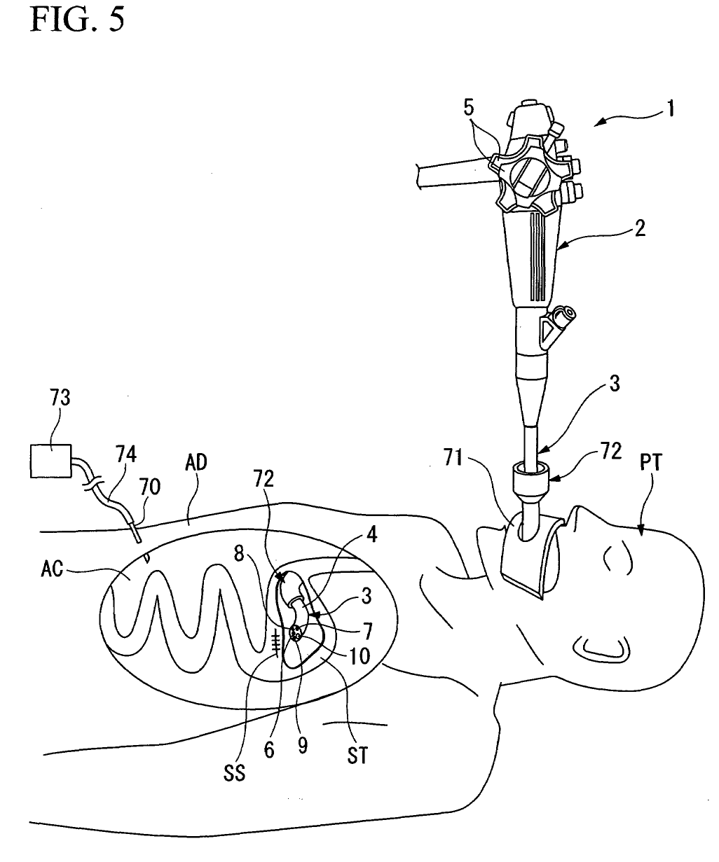

Once the inserted part is withdrawn back into the stomach, the opening SO in the stomach wall is closed by suturing closed using a suturing device. As a result, as shown in FIG. 5, a sutured area SS (closed area) is formed at the site where the opening SO was located.

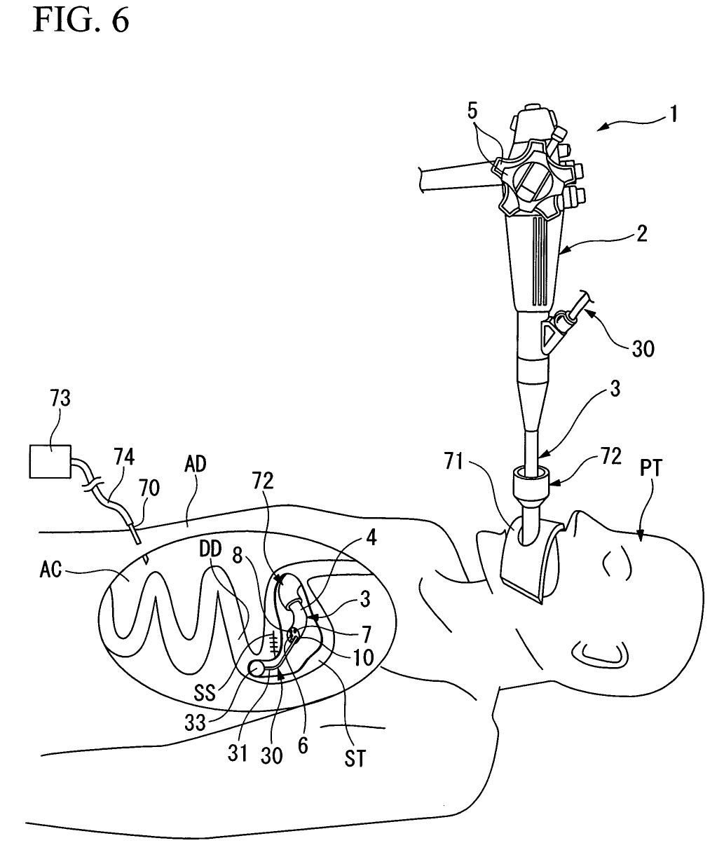

Next, a leak test of the sutured site SS is carried out. First, the suturing device is removed from the inside of the body, and a balloon 30 is introduced into the body. The bougie 33 is disposed to the entrance into the duodenum, a syringe is attached to the cap 32 on the operator's side, and fluid is supplied to the sheath 31 to inflate the bougie 33. As shown in FIG. 6, when the bougie 33 is inflated, it adheres closely to the inside surface of the stomach wall, thereby closing the entrance to the duodenum DD.

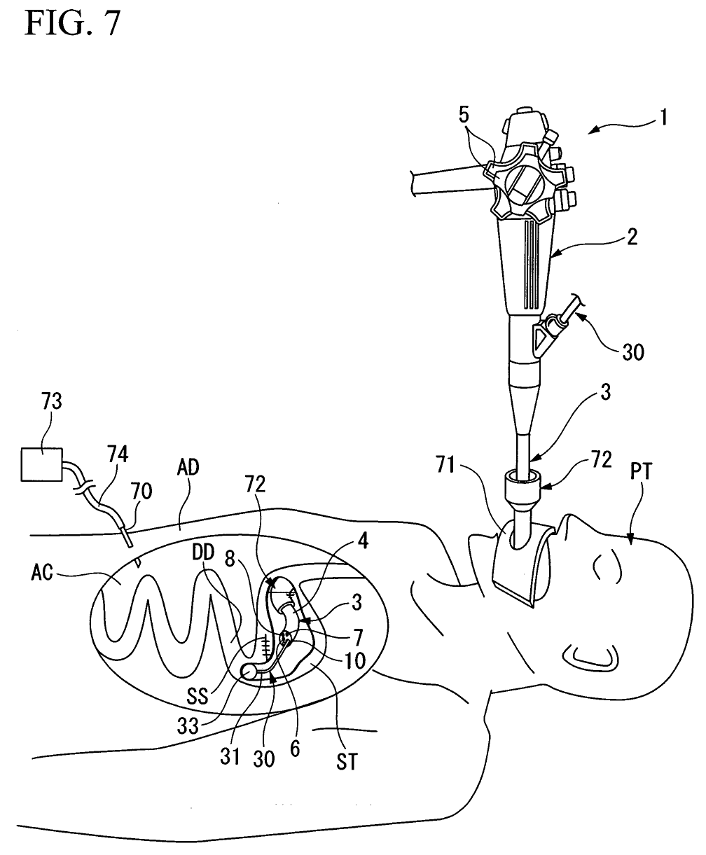

Further, as shown in FIG. 7, water is introduced from the water supply device 21 through the gas-and-water introducing channel 8, to supply water into the stomach ST. At least enough water is supplied to cover over the suture site SS, and it is acceptable to fill the inside of the stomach ST with water. However, it is not absolutely essential to apply pressure with the water.

In this state, air is supplied into the abdominal cavity AC via the inflating needle 70, and the sutured site SS is observed with the observation device 6 of the endoscope 1, which is a device for observation and viewing. The pressure of the air may be of a degree that is typically employed when infusing air into the abdomen. When the opening at the suture site SS is completely closed, air from the abdominal cavity AC can not pass through the sutured site SS. Accordingly, no change should be seen. In contrast, when the suture site SS is not completely closed, then air from the abdominal cavity AC can pass through the suture site SS, and enter into the stomach ST. As a result, bubbling from the suture site SS will occur. If bubbling is confirmed with the observation device 6, then the opening is incompletely closed. Accordingly, the water within the stomach ST is suctioned out using the suction channel 9 of the endoscope 6, and the procedure to close the opening is repeated. The procedures up until this point in time are repeated until no further bubbling from the suture site SS occurs.

Once there is no further bubbling from the suture site SS, the water is removed from the stomach ST using the suction channel 9, the bougie 33 of the balloon 30 is deflated, and the endoscope 1 is removed from patient ST. Next, the air inflating the abdominal cavity AC is expelled via the inflating needle 70, and the inflating needle 70 is removed.

With conventional techniques, it was difficult to confirm that the opening was closed using just the observation device 6 of the endoscope 1. In the present embodiment, however, water is supplied to the stomach ST, while air is supplied on the abdominal cavity AC side. As a result, it is possible to easily confirm simply by the presence or absence of bubbling that the stomach ST is closed at the suture site SS. As a result, the procedure is completed with only a small hole for the inflating needle 70 formed in the abdominal wall AW, without requiring insertion of instruments from the abdominal wall AW. Thus, since no great injury is caused to the patient PT, recovery is hastened.

It is not necessary to insert a particular instrument in order to confirm that the opening is closed. Thus, the confirming operation can be carried out quickly and with good efficiency.

The following two approaches may be employed in place of insertion of the inflating needle 70 into the abdominal wall AW, as methods for inflating the abdominal cavity AC by introducing air.

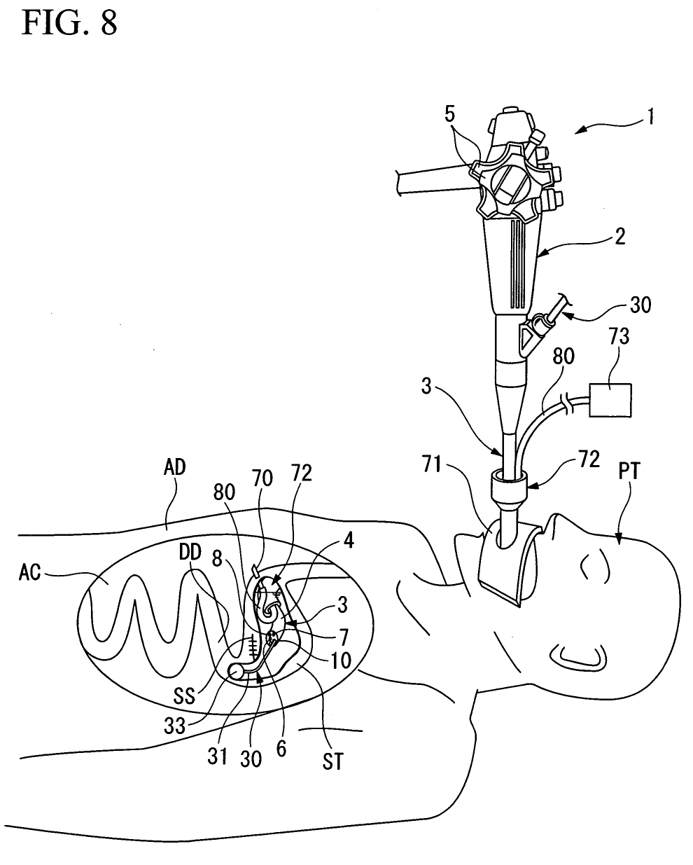

As shown in FIG. 8, abdominal cavity AC can be inflated by inserting inflating needle 70 into the abdominal cavity AC from within the stomach ST. An inflating tube 80 for introducing gas is connected to inflating needle 70. The inflating tube 80 is passed through the stomach and esophagus and is pulled out from the mouth of the patient PT, and is connected to a gas inflating device 73. A gas such as carbon dioxide gas which is supplied from the gas inflating device 73 passes through each of the conduits of inflating tube 80 and inflating needle 70, and inflates the abdominal cavity AC. The inflating tube 80 is passed through over-tube 72. However, in the case where the endoscope 1 has two or more working channels 10, then it may be passed through the endoscope 1. Once the leak test is completed, the air inside the abdominal cavity AC is passed through the inflating needle 70 and expelled, after which inflating needle 70 is withdrawn from the stomach ST. The inflating needle 70 is narrow, so that the hole formed by its passage is small. The area through which the inflating needle 70 is passed quickly heals. In this modification, it is not necessary to form an opening in the abdominal wall AW. Accordingly, this modification is more preferable from the perspective of patient esthetics and injury recovery. The other effects in this modification are equivalent to those described above.

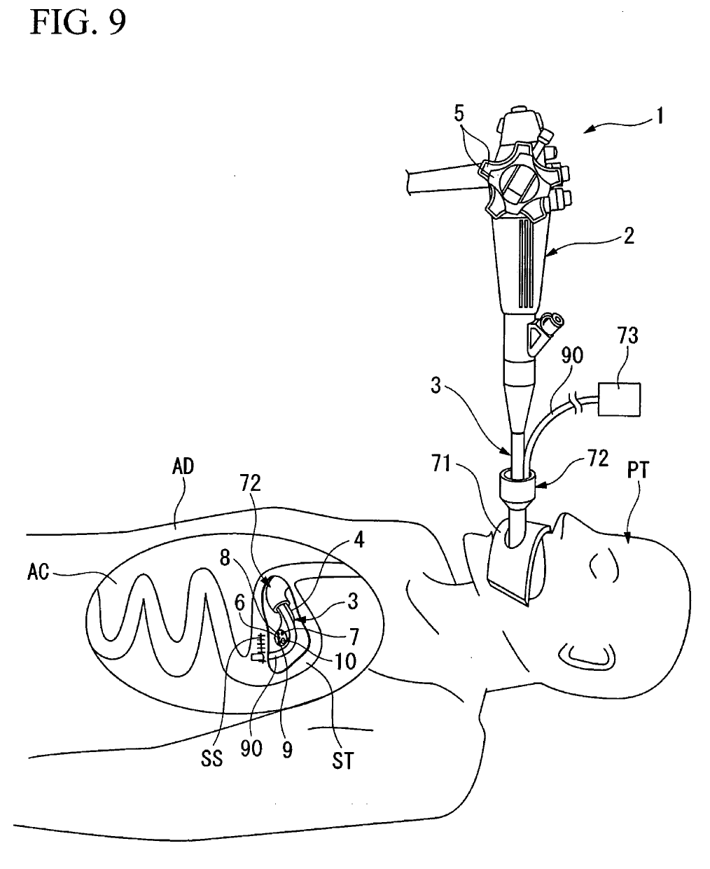

As shown in FIG. 9, it is also acceptable to pass testing tube 90 through the suture site SS. The testing tube 90 is a flexible narrow tube. When closing the opening, suturing is performed with the testing tube 90 remaining passed through the opening. When air is introduced by passing it through the conduit of the testing tube 90, the abdominal cavity AC can be inflated. The testing tube 90 is passed through the over-tube 72. However, in the case where the endoscope 1 has two or more working channels 10, then it may be passed through the endoscope 1. Once the leak test is completed, the air inside the abdominal cavity is passed through the testing tube 90 and expelled, after which the testing tube 90 is withdrawn from the suture site SS. The testing tube 90 is narrow, so that the area through which it is passed does not effect closing the opening, and healing occurs quickly. In this modification, the number of openings formed in the stomach ST and the abdominal wall AW can be reduced, so that the burden on the patient PT can be lessened even further and a faster recovery can be anticipated. The other effects in this modification are equivalent to those described above.

### Second Embodiment

A second embodiment of the present invention will now be described in detail with reference to the accompanying figures.

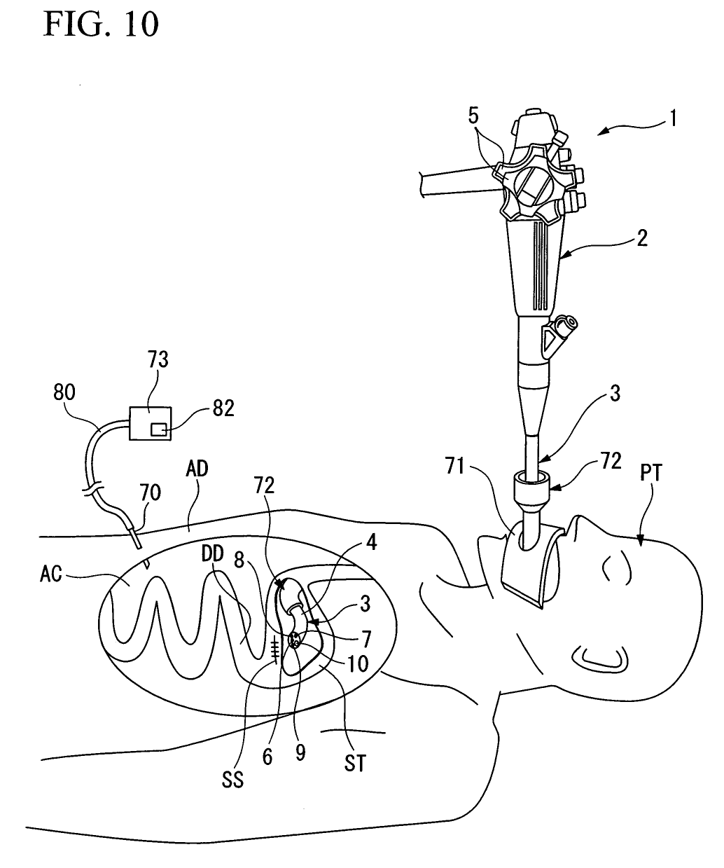

As shown in FIG. 10, the inflating needle 70 is connected to an inflating device 73 via an inflating tube 80, and a pressure sensor 82 is attached that can detect the pressure inside inflating needle 70. Note that in this embodiment, the inflating needle 70, the inflating tube 80 and the inflating device 73 are employed as the observation device.

When carrying out a medical procedure, air is supplied from the inflating needle 70 to inflate the abdominal cavity AC. Next, the inserted part 3 is passed through the opening SO formed in the stomach ST, and is introduced into the abdominal cavity AC, where the necessary procedure is then carried out. The inserted part 3 is then withdrawn into the stomach ST, and the opening is closed. The process up until this point in time is the same as in the first embodiment.

When performing the leak test, the gas employed to inflate the stomach ST is evacuated from suction channel 9 of the endoscope 1, and a pressure difference is provided between the pressure within the stomach ST and the pressure within the abdominal cavity AC. Next, the introduction of gas from the inflating needle 70 is stopped, and the change in pressure within the abdominal cavity AC is checked with a pressure sensor 82. Since air, which is less readily absorbed by the body than carbon dioxide, was used as the inflating gas, there should be almost no change in the pressure within the abdominal cavity AC if the closure in the stomach ST at the suture site SS is certain. In contrast, if the closure in the stomach ST is not complete, the air will leak into the stomach ST where the pressure is lower, and the pressure detected at the pressure sensor 82 will fall. In this case, the suture site SS must be resutured so that there is almost no drop in pressure within the abdominal cavity AC. Once the drop in pressure has been almost entirely eliminated, the endoscope 1 is withdrawn from the patient PT. Air is evacuated from the abdominal cavity AC and the inflating needle 70 is withdrawn.

Note that data for the standard pressure drop in the abdominal cavity AC is recorded in advance in the control device 24. By making a decision about leaking at the suture site SS by comparing with this data, a quick and certain decision can be made. By incorporating the use of this kind of data, it is possible to carry out the leak test with surety, even using carbon dioxide which is absorbed comparatively more quickly.

In this embodiment, a leak test of the suture site SS of the stomach ST was carried out by monitoring the pressure of the gas supplied to the abdominal cavity AC from the inflating needle 70. As a result, the same effects as obtained in the first embodiment are obtained. In particular, by employing a pressure sensor 82 at the inflating needle 70, the leak test can be performed without using the endoscope 1. For this reason, the leak test can be carried out after removing the endoscope 1 from the patient. In this case, the stress on the patient PT can be reduced. Further, when the leak test is carried out while observing the suture site SS with the endoscope 1, it is possible to confirm a leak with even greater certainty. In this case, it is acceptable to supply water into the stomach ST.

Note that in this embodiment it is also possible to employ a modification such as shown in FIG. 8 or FIG. 9 in place of piercing the inflating needle 70 into the abdominal wall AW.

The preferred embodiments have been described above, however, the present invention is not limited thereto. Rather, additions, omissions, substitutions and other variations to the design are possible as long as they do not depart from the spirit of the present invention. The present invention should not be limited to the above descriptions, but rather is limited only by the appended claims.

For example, in the case where the operating channel 10 of the endoscope 1 has a design the enables connection to the water introducing device 21 or the gas introducing device 22, it is acceptable to introduce gas or fluid using the operating channel 10.

It is acceptable to change the inclination of the patient PT's body according to the position where the opening SO is formed so that the suture site SS is submerged in water. The intention is to enable a leak test with a small amount of water and to reduce the testing time. In particular, when the bed on which the patient PT is laying is raised up, changing the inclination of the body so that the suture site SS is directed downward, it is possible to carry out an accurate test with a small amount of water.

When water is introduced into the stomach ST, it is acceptable to dispose a balloon 30 near the cardia on the esophageal side of the stomach ST, and inflate the bougie 33. This prevents the outflow of water with even greater certainty, and enables the leak test to be carried out quickly.

The device required to carry out the desired procedure within the abdominal cavity AC is not limited to an endoscope equipped the observation device and operating channel as disclosed in the preceding embodiments. For example, a device may be employed that has a treatment part for performing the desired treatment provided to the end of the inserted part that is inserted into the body, and is provided with an operating part that enables this treatment part to be manipulated from outside the body. In this case, the medical procedure is carried out while observing with the observation device that can be swallowed, such as a capsule endoscope.

An observation device that can be swallowed, such as a capsule endoscope, can be used as the observation device that observes the presence or absence of bubbles occurring along the suture site SS. The conduit for supplying and evacuating a fluid into and from the stomach ST, which is the hollow organ, is not limited to the endoscope 1. It is also acceptable to use a device that does not have an operating channel 10 or the observation device 6.

The hollow organ in which opening SO is formed is not limited to the stomach ST. Other examples of hollow organs that are acceptable include the esophagus, duodenum, small bowel, large bowel, uterus, bladder or the like.

In addition, suturing was disclosed as an example of a means for closing the opening that communicates between the hollow organ and the abdominal cavity, however, the present invention is not limited thereto. For example, it is also acceptable to close the communication between the hollow organ and the abdominal cavity by anastamosis, for example.

1. A leak test method for a medical procedure via a natural orifice, comprising:
inflating an abdominal cavity by introducing a gas into the abdominal cavity via a conduit inserted from the abdominal cavity after closing an opening that communicates between the inside of a hollow organ and the inside of the abdominal cavity using an instrument inserted via the patient's natural orifice;
monitoring no leakage between the hollow organ and the abdominal cavity of a fluid passing through the closed opening; and
removing the conduit inserted into the abdominal cavity once no leakage of the fluid has been confirmed.

2. The method according to claim 1, wherein inflating the abdominal cavity comprises inserting an inflating needle having a conduit through an abdominal wall into the abdominal cavity.

3. The method according to claim 2, wherein monitoring comprises inserting an observation device via the natural orifice, and confirming the presence or absence of a flow of the fluid by the observation device.

4. The method according to claim 2, wherein monitoring comprises detecting a pressure inside the abdominal cavity via the inflating needle.

5. The method according to claim 1, wherein inflating the abdominal cavity comprises inserting an inflating needle having a conduit into the hollow organ via the natural orifice, and piercing the inflating needle into the abdominal cavity from inside the hollow organ.

6. The method according to claim 5, wherein monitoring comprises inserting the observation device via the natural orifice, and confirming the presence or absence of a flow of the fluid with the observation device.

7. The method according to claim 5, wherein monitoring comprises detecting a pressure inside the abdominal cavity via the inflating needle.

8. The method according to claim 1, wherein inflating the abdominal cavity comprises closing the opening with the tube while passing a tube through the opening in place, and supplying gas to the abdominal cavity from the tube.

9. The method according to claim 8, wherein monitoring comprises inserting the observation device via the natural orifice, and confirming the presence or absence of a flow of the fluid with the observation device.

10. The method according to claim 8, wherein monitoring comprises detecting a pressure inside the abdominal cavity via the inflating needle.

11. The method according to claim 1, further comprising supplying the fluid to the hollow organ having the closed opening.

12. The method according to claim 11, further comprising varying the body position of the patient so that the closed opening is submerged in the fluid.
